(19) United States
(12) Patent Application Publication
(10) Pub. No.: US 2008/0008745 A1
(43) Pub. Date: Jan. 10, 2008

(54) Transdermal delivery of naltrexone hydrochloride, naltrexol hydrochloride, and bis(hydroxy-methyl)propionyl-3-0 ester naltrexone using microneedles

(75) Inventors: Audra Lynn Stinchcomb, Lexington, KY (US); Stan Lee Banks, Lexington, KY (US); Raghotham Reddy Pinninti, Lexington, KY (US)

Correspondence Address: BUCHANAN, INGERSOLL & ROONEY PC, POST OFFICE BOX 1404, ALEXANDRIA, VA, 22313-1404, US

(73) Assignee: UNIVERSITY OF KENTUCKY RESEARCH FOUNDATION, Lexington, KY (US)

(21) Appl. No.: 11/812,249

(22) Filed: Jun. 15, 2007

Related U.S. Application Data
(60) Provisional application No. 60/815,256, filed on Jun. 21, 2006.
(60) Provisional application No. 60/903,285, filed on Feb. 26, 2007.

(51) Int. Cl. A61K 31/4355; A61K 9/00; A61K 9/70; A61P 25/32; A61P 25/36

(52) U.S. Cl. 424/447; 424/400; 424/443; 514/282

(57) ABSTRACT

The present invention provides methods for transdermal delivery of a therapeutically effective amount of Naltrexone Hydrochloride, Naltrexol Hydrochloride, and/or Naltrexone Diol Ester using microneedles. The invention also provides methods for treatment of narcotic dependence, alcohol abuse, and/or alcoholism. Preferably, the Naltrexone Hydrochloride, Naltrexol Hydrochloride, and/or Naltrexone Diol Ester is administered by creating a microneedle-treated site in the skin of a subject by inserting microneedles, followed by applying the Naltrexone Hydrochloride, Naltrexol Hydrochloride, and/or Naltrexone Diol Ester to the microneedle-treated site.

### GOVERNMENT INTERESTS

This invention was made with government support under NIH Grant Number RO1DA13425. The Government may have certain rights in this invention.

### BACKGROUND OF THE INVENTION

1. Technical Field

The present invention relates generally to the transdermal delivery of Naltrexone Hydrochloride, Naltrexol Hydrochloride, and a diol ester of Naltrexone (bis(hydroxymethyl)propionic acid ester prodrug of naltrexone, or bis(hydroxymethyl)propionyl-3-O-ester naltrexone) using microneedles. More particularly, the present invention relates to methods of using microneedles to facilitate transdermal delivery of a therapeutically effective amount of Naltrexone Hydrochloride, Naltrexol Hydrochloride, and/or Naltrexone Diol Ester for treatment of narcotic dependence, alcohol abuse, and alcoholism.

2. Background Art

The treatment of those affected by alcohol and drug abuse is of high priority. It was estimated by Mark et al. that spending for mental health and drug abuse grew from $60 billion in 1991 to $104 billion in 2001 (Mark et al., Health Aff (Millwood) Suppl. Web Exclusives: W5-133-W5-142, 2005). The economic cost to society from alcohol abuse and alcoholism was estimated at $148 billion, and an additional $98 billion was attributed to drug abuse in 1992 (Harwood et al., Addiction 94 (1999) 631-635). At such high costs to society, efforts must be coordinated to provide sufferers with safe and effective therapies.

Current therapeutics available to patients who suffer from addiction include methadone and buprenorphine treatment, as well as Naltrexone. However, methadone treatment requires expert supervision in a clinical setting, and buprenorphine has a complicated and clinician-supervised titration dosing regimen.

Naltrexone is currently available as REVIA®, an FDA approved 50 mg tablet of Naltrexone Hydrochloride, and as VIVITROL™, the recently FDA approved 28-day controlled release 380 mg depot form of Naltrexone. However, REVIA® is poorly bioavailable, with documented side effects (PDR, Medical Economics, 1996, 2229-2233, New Jersey). In addition, although long-lasting Naltrexone depot formulations have shown plasma levels for up to 30 days (Galloway et al., BMC Psychiatry 5 (2005) 18), once VIVITROL™ is injected, it cannot be easily discontinued without painful surgical removal. Thus, there is a need for methods for transdermally transporting a therapeutically effective amount of Naltrexone, in order to have an outpatient therapy option that provides controlled release, reduced side effects, and the ability to readily discontinue therapy.

The main challenge in transdermal drug delivery is providing sufficient drug penetration across the skin. A large majority of drugs, including Naltrexone, are unable to cross the skin at therapeutic rates due to the barrier imposed by the skin's outer stratum corneum layer. Stinchcomb and colleagues have developed prodrug derivatives of Naltrexone in an attempt to increase the permeability of this opioid antagonist (Hammell et al., J. Control. Release 97 (2004) 283-290; Hammell, J. Pharm. Sci. 94 (2005) 828-836; Nalluri et al., Drug Dev. Ind. Pharm. 31 (2005) 871-877; Paudel et al., J. Pharm. Sci. 94 (2005) 1965-1975; and Pillai et al., Pharm. Res. 21 (2004) 1146-1152). Although many of the 3-O-alkyl ester and carbonate prodrugs of Naltrexone showed significant permeability enhancement over Naltrexone, permeation levels were still lower than desired (Id.).

Skin permeability can be increased through the use of chemical enhancers, electrical enhancers via electroporation or iontophoresis, ultrasonic enhancers, and a variety of other approaches. Although these enhancement technologies are still under active investigation, delivering macromolecules into the skin remains a significant challenge. See, e.g., Park et al., J. Controlled Release 104 (2005) 51-66 and Martanto et al., Pharm. Res. 21 (2004).

An alternative approach to increase transdermal transport involves using arrays of microscopic needles (or “microneedles”) to pierce the skin, thus creating micrometer-scale transport pathways. Microneedles provide a minimally invasive means to transport molecules into the skin, as the channels they create are extremely small on a clinical level. However, because the channels are much larger than macromolecules, such channels should dramatically increase skin permeability. Id.

Currently, microneedles are made from silicon, biodegradable polymers, and stainless steel. Microneedles can be solid or hollow. Solid microneedles can be used to create holes in the skin, followed by application of a transdermal patch to the skin surface. Alternatively, solid microneedles can be first coated with a drug and then inserted into the skin. Hollow microneedles can also be used, to facilitate active fluid flow through the needle bore and into the skin. See, e.g., Prausnitz, Adv. Drug. Deliv. Rev. 56 (2004) 581-587, for a review.

Numerous studies have demonstrated that solid microneedles can increase skin permeability by up to four orders of magnitude for compounds ranging in size from small molecules to proteins to nanoparticles (Henry et al., J. Pharm. Sci. 87 (1988) 922-925; McAllister et al., PNAS 100 (2003) 13755-13760; Lin et al., Pharm. Res. 18 (2001) 1787-1793; and Cormier et al., J. Control. Release. 97 (2004) 503-511). Hollow microneedles have also been shown to deliver insulin and reduce blood glucose levels (McAllister et al., PNAS 100 (2003) 13755-13760; Martanto et al., Pharm. Res. 21 (2004) 947-952). Kaushik et al. studied the effects of pain associated with microneedle insertion in human volunteers and showed that the sensation was no more than that of a smooth surface applied to the skin or the “sensation of a piece of tape” applied to the skin (Kaushik et al., Anesth. Analg. 92 (2004) 502-504).

### SUMMARY OF THE INVENTION

The use of salts and ionized drugs is not typically optimal for standard passive transdermal dosage forms where the main route of diffusion is through the lipid bilayers of the stratum corneum. However, transport across skin treated with microneedles occurs through aqueous channels. The present inventors discovered that the water soluble salts and diol ester forms of Naltrexone and Naltrexol are capable of permeating the skin via diffusion through the aqueous conduits created by microneedle skin treatment. Accordingly, the present invention overcomes the problems associated with existing delivery systems for Naltrexone by providing methods for transdermal delivery of a therapeutically effective amount of Naltrexone Hydrochloride, Naltrexol Hydrochloride, and/or Naltrexone Diol Ester.

A first aspect of the invention provides a method for transdermal delivery of Naltrexone Hydrochloride, Naltrexol Hydrochloride, and/or Naltrexone Diol Ester, wherein said method comprises the steps of (a) creating a microneedle-treated site in the skin of a subject by inserting mironeedles into the skin of said subject, followed by (b) applying said Naltrexone Hydrochloride, Naltrexol Hydrochloride, and/or Naltrexone Diol Ester to said microneedle-treated site.

A second aspect of the invention provides a method for treating narcotic dependence, alcohol abuse, and/or alcoholism, comprising administering to a subject in need thereof a therapeutically effective amount of Naltrexone Hydrochloride, Naltrexol Hydrochloride, and/or Naltrexone Diol Ester, wherein said Naltrexone Hydrochloride, Naltrexol Hydrochloride, and/or Naltrexone Diol Ester is administered transdermally using microneedles.

Preferably, the method for treating narcotic dependence, alcohol abuse, and/or alcoholism comprises administering to a subject in need thereof a therapeutically effective amount of Naltrexone Hydrochloride, Naltrexol Hydrochloride, and/or Naltrexone Diol Ester, wherein said Naltrexone Hydrochloride, Naltrexol Hydrochloride, and/or Naltrexone Diol Ester is administered transdermally using microneedles, and wherein said method comprises the steps of (a) creating a microneedle-treated site in the skin of said subject by inserting microneedles into the skin of said subject, followed by (b) applying said Naltrexone Hydrochloride, Naltrexol Hydrochloride, and/or Naltrexone Diol Ester to said microneedle-treated site.

In an embodiment of the invention, the Naltrexone Hydrochloride, Naltrexol Hydrochloride, and/or Naltrexone Diol Ester is applied to said microneedle-treated site via a gel or a hydrogel formulation.

The present invention further provides gel and hydrogel formulations comprising Naltrexone Hydrochloride, Naltrexol Hydrochloride, and/or Naltrexone Diol Ester.

### BRIEF DESCRIPTION OF THE DRAWINGS

FIG. 1. shows the structures of Naltrexone base (a), Naltrexone HCl (b), and 6-β-Naltrexol at pH 8.5 (c) and 4.5 (d).

FIG. 2. shows permeation and steady state flux profiles of microneedle treated guinea pig skin with Naltrexone (NTX) base (♦) and hydrochloride (▪).

FIG. 3. shows permeation and steady state flux profiles of untreated guinea pig skin with Naltrexone base (♦) and hydrochloride (▪) (p<0.05).

FIG. 4. shows permeation and steady state flux profiles of human skin with Naltrexone hydrochloride in presence (▪) or absence (♦) of MN's (p<0.05).

FIG. 5. shows permeation and steady state flux profiles of microneedle treated guinea pig skin with Naltrexol (NTXOL) base pH 8.5 (x) and untreated GP skin with Naltrexol base pH 8.5 (▴) and 4.5 (♦).

FIG. 6. shows permeation and steady state flux profiles of microneedle treated guinea pig skin with Naltrexol base, pH 4.5 (▪) and 8.5 (▴).

FIG. 7. shows the synthesis of bis(hydroxymethyl)propionyl-3-O ester naltrexone (4).

FIG. 8. shows a representative plasma profile comparing Naltrexol HCL and microneedle treatment with Naltrexol base and no microneedle treatment in guinea pigs.

FIGS. 9-12 show the plasma profiles of NTX in microneedle treated human subjects.

FIG. 13. shows the average plasma profile of NTX in microneedle treated human subjects.

FIG. 14. shows the average plasma profile of NTXOL in microneedle treated human subjects.

FIG. 15. shows the average plasma profile of NTX and NTXOL in microneedle treated human subjects.

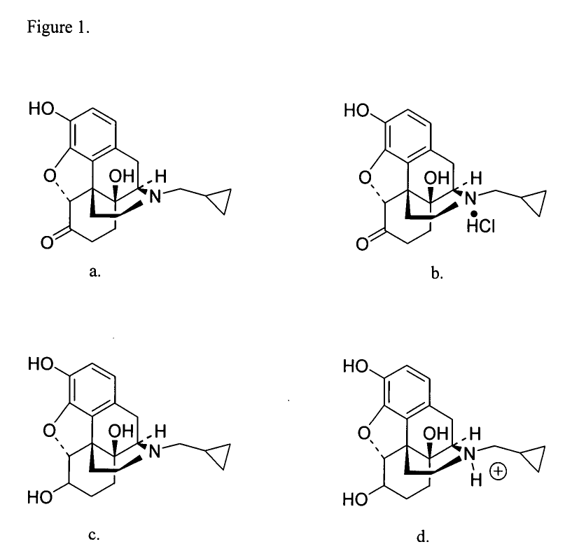

### DETAILED DESCRIPTION OF THE INVENTION

As noted above, the present invention relates to methods for treating narcotic dependence and alcohol addiction. Subjects who can benefit from the methods of the present invention include, for example, mammals, such as humans, particularly humans who are suffering from narcotic dependence, alcohol abuse, and/or alcoholism.

“Treatment” or “treating,” as used herein, refers to complete elimination as well as to any clinically or quantitatively measurable reduction in the subject's narcotic dependence, alcohol abuse, and/or alcoholism.

The methods of the present invention involve transdermally delivering a therapeutically effective amount of Naltrexone Hydrochloride, Naltrexol Hydrochloride, and/or Naltrexone Diol Ester. A “therapeutically effective amount,” as used herein, refers to a transdermally delivered amount, determined by one skilled in the art, sufficient for treating the subject's narcotic dependence, alcohol abuse, and/or alcoholism.

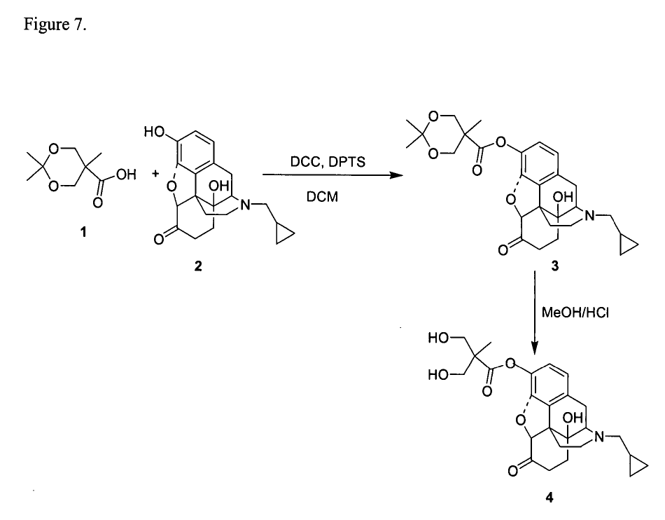

The “diol ester of Naltrexone” or “Naltrexone Diol Ester,” as used herein, refers to the bis(hydroxymethyl)propionic acid ester or 3-hydroxy-2-hydroxymethyl-2-methyl-propionic acid ester prodrug of naltrexone, or bis(hydroxymethyl)propionyl-3-O-ester naltrexone, as shown in FIG. 7 of the present application.

The Naltrexone Hydrochloride, Naltrexol Hydrochloride, and/or Naltrexone Diol Ester is preferably administered as a pharmaceutical composition comprising pharmaceutically acceptable carriers, diluents, and/or excipients, which are vehicles commonly used to formulate pharmaceutical compositions for animal or human administration. The carriers, diluents and/or excipients are not intended to have biological activity themselves, and are selected so as not to affect the biological activity of the Naltrexone Hydrochloride, Naltrexol Hydrochloride, and/or Naltrexone Diol Ester and any other active agent(s). A pharmaceutically acceptable carrier, diluent, and/or excipient as used herein includes both one and more than one such carrier, diluent, and/or excipient. Examples include but are not limited to distilled water, saline, physiological phosphate-buffered saline, Ringer's solutions, dextrose solution, and Hank's solution.

Depending upon the manner of introduction, the Naltrexone Hydrochloride, Naltrexol Hydrochloride, and/or Naltrexone Diol Ester may be formulated as, for example, a sterile injectable formulation comprising aqueous solutions and/or suspensions containing the active materials in admixture with suitable carriers, diluents, and/or excipients.

The concentration of Naltrexone Hydrochloride, Naltrexol Hydrochloride, and/or Naltrexone Diol Ester the formulation (i.e., a formulation that is therapeutically effective to the subject to which it is administered) and the dose administered can be readily determined by a person of ordinary skill in the art. Typically, dosages used in vitro and in animal models, such as in the experiments provided in the present application, may provide useful guidance in the amounts useful for in vivo administration.

The methods of the present invention involve transdermal delivery using microneedles. The microneedle strategy suitable for use in the present invention is not particularly limited, but includes any microneedle strategy that can be employed to facilitate the transdermal delivery of Naltrexone Hydrochloride, Naltrexol Hydrochloride, and/or Naltrexone Diol Ester.

As used herein, the term “microneedles” refers to a plurality of elongated structures that are sufficiently long to penetrate through the stratum corneum skin layer and into the epidermal/dermal layer, yet are also sufficiently short to not activate nerve endings and cause pain.

Various microneedles that can be employed to facilitate transdermal delivery of drugs such as Naltrexone Hydrochloride, Naltrexol Hydrochloride, and/or Naltrexone Diol Ester are reviewed in Prausnitz, Advanced Drug Delivery Reviews 56 (2004) 581-587. See also Zahn et al., Biomed. Microdevices 6 (2004) 183-190; Shirkhandeh J. Materials Sci. 16 (2005) 37-45; Park, J. Controlled Release 104 (2005) 51-66; U.S. Pat. Nos. 3,964,482, 6,503,231, 6,745,211; 6,611,707; 6,334,856; and U.S. Published Patent Application Nos. 2005/0,209,565, 2004/0,106,904, 2004/0,186,419, and 2002/0,193,754. Suitable microneedles have been fabricated from many materials, including silicon, metals, and polymers. Further, Davis et al. have studied the mechanics of microneedle insertion into the skin (Davis et al., J. Biomech. 37 (2004) 1155-1163).

The microneedles can be solid or hollow. If solid microneedles are used, channels can be made by poking the skin with a microneedle array, followed by removal of the needles and then application of the drug (see, e.g., Martanto et al., Pharm. Res. 21 (2004) and McAllister et al., PNAS 100 (2003) 13755-13760). Alternatively, the solid microneedles can be inserted and left in the skin, allowing diffusion through the gaps between the microneedles and the surrounding tissue (McAllister et al., PNAS 100 (2003)). Solid microneedles can also be coated with the drug prior to insertion into the skin, in which case all the drug to be delivered is on the needle itself and no drug reservoir on the skin surface is needed (Prausnitz, Advanced Drug Delivery Reviews 56 (2004) 581-587). Alternatively, microneedles containing a hollow bore can be used to transport drugs through the interior of the needles by diffusion or pressure-driven flow (Id.). Hollow microneedles can act as mini hypodermic injection needles (Zahn et al., Biomed. Microdevices 6 (2004) 183-190).

Preferably, the microneedles of the present invention are used to create a microneedle-treated site prior to applying the Naltrexone Hydrochloride, Naltrexol Hydrochloride, and/or Naltrexone Diol Ester to the microneedle-treated site. As noted above, the microneedles can be inserted and left in the skin. More preferably, a microneedle array comprised of solid microneedles is used to increase skin permeability by inserting the microneedles into the skin and then removing the microneedles to create a microneedle-treated site prior to applying the Naltrexone Hydrochloride, Naltrexol Hydrochloride, and/or Naltrexone Diol Ester to the microneedle-treated site. Optionally, the microneedles can be repeatedly inserted and removed, at the same site (Martanto et al., Pharm. Res. 21 (2004). Typical microneedle arrays comprise 15-200 microneedles. A preferred microneedle array comprises 50-100 microneedles.

In practice, the microneedles are inserted and removed to create a microneedle-treated site on the subject's skin under conditions effective to transdermally deliver the Naltrexone Hydrochloride, Naltrexol Hydrochloride, and/or Naltrexone Diol Ester upon application of the Naltrexone Hydrochloride, Naltrexol Hydrochloride, and/or Naltrexone Diol Ester. Such conditions can include, for example, positioning the microneedle-treated site on a portion of the subject's skin which is not covered with hair, and/or shaving the hair from the selected portion of the subject's skin.

The Naltrexone Hydrochloride, Naltrexol Hydrochloride, and/or Naltrexone Diol Ester can be applied to the microneedle-treated site as a gel, hydrogel, topical cream, salve, ointment, or other topical formulation; and/or by using delivery devices such as bandages, occlusive bodies, patches, and/or the like.

Illustratively, a Naltrexone Hydrochloride, Naltrexol Hydrochloride, and/or Naltrexone Diol Ester composition that is applied to the microneedle-treated site can be formulated as a gel, hydrogel, topical cream, salve, or ointment. The topical formulations can include inert diluents and carriers as well as other conventional excipients, such as wetting agents, preservatives, and suspending and dispersing agents. In addition to the above, generally non-active components, topical formulations containing Naltrexone Hydrochloride, Naltrexol Hydrochloride, and/or Naltrexone Diol Ester can further include other active materials, particularly, active materials which have been identified as useful in the treatment of drug and alcohol addiction and which can usefully be delivered transdermally to the subject. For instance, such other active materials can include acamprosate, disulfiram, topiramate, sertraline, rivastigmine, citalopram, and doxepin. The topical formulation can be applied directly to the skin and then optionally covered (e.g., with a patch or bandage of gauze) to minimize the likelihood of its being disturbed. Alternatively, the topical formulation can be coated on the surface of a patch, bandage, gauze, etc., and the patch, bandage, gauze, etc. can then be applied to the skin of the subject such that the topical formulation is in direct contact with the subject's skin.

Preferably, the Naltrexone Hydrochloride, Naltrexol Hydrochloride, and/or Naltrexone Diol Ester is applied to the skin in the form of a gel, such as a hydrogel.

As used herein, the term “gel” or “gel matrix” means a type of reservoir or vehicle for Naltrexone Hydrochloride, Naltrexol Hydrochloride, and/or Naltrexone Diol Ester. A gel takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles), or a network of polymer chains. In some embodiments, a gel matrix may include hydrogels, organogels, and the like.

A “hydrogel” or “hydrogel matrix” refers to a three dimensional network of, for example, cross-linked hydrophilic polymers in the form of a gel and substantially composed of water.

Protocols for forming gels, including hydrogels, are well known in the art, as are protocols for forming gels and hydrogels comprising therapeutically effective amounts of one or more active agents.

A preferred gel formulation comprises about 5 to about 30 weight % Naltrexone Hydrochloride, Naltrexol Hydrochloride, and/or Naltrexone Diol Ester; about 5 to about 50 weight % water; about 30 to about 70 weight % propylene glycol; and about 1 to about 5 weight % hydroxyethylcellulose polymer. More preferably, the gel comprises about 15 to about 20 weight % Naltrexone Hydrochloride, Naltrexol Hydrochloride, and/or Naltrexone Diol Ester; about 15 to about 20 weight % water; about 50 to about 60 weight % propylene glycol; and about 2.0 weight % hydroxyethylcellulose polymer. The gel can optionally include a preservative, such as benzyl alcohol, propylparaben, and/or methylparaben.

A preferred hydrogel formulation comprises about 15 to about 20 weight % Naltrexone Hydrochloride, Naltrexol Hydrochloride, and/or Naltrexone Diol Ester; about 10 to about 40 weight % water; about 35 to about 70 weight % poly(propylene glycol) acrylate, (poly(ethylene glycol) methacrylate, and/or (poly(ethylene glycol) dimethacrylate; and about 0.5 to about 5 weight % polyvinylpyrrolidone. The hydrogel can optionally include a preservative, such as benzyl alcohol, propylparaben, and/or methylparaben.

In a preferred embodiment a bandage, pad, or other type of patch can be placed over the drug formulation, for example a gel or hydrogel formulation comprising Naltrexone Hydrochloride, Naltrexol Hydrochloride, and/or Naltrexone Diol Ester.

Alternatively, or in addition, the Naltrexone Hydrochloride, Naltrexol Hydrochloride, and/or Naltrexone Diol Ester can be delivered transdermally to the subject by formulating Naltrexone Hydrochloride, Naltrexol Hydrochloride, and/or Naltrexone Diol Ester into a bandage, pad, or other type of patch which can be applied to the subject's skin.

Illustratively, matrix-type transdermal patches, in which the Naltrexone Hydrochloride, Naltrexol Hydrochloride, and/or Naltrexone Diol Ester is disposed in an adhesive matrix, can be employed. The matrix-type transdermal patch can further include other active materials for transdermal delivery to the subject with the Naltrexone Hydrochloride, Naltrexol Hydrochloride, and/or Naltrexone Diol Ester. Suitable adhesives for use in such matrix-type transdermal patches include polyisobutylenes, acrylates, silicone, and combinations thereof. Still other patches suitable for use in the practice of the present invention include those described in U.S. Pat. No. 5,223,262.

In another illustrative embodiment, the bandage, pad, or other type of patch can be one which is capable of controlling the release of the Naltrexone Hydrochloride, Naltrexol Hydrochloride, and/or Naltrexone Diol Ester such that transdermal delivery of the Naltrexone Hydrochloride, Naltrexol Hydrochloride, and/or Naltrexone Diol Ester to the subject is substantially uniform and sustained over a period of at least 12 hours, such as at least 24 hours, at least 48 hours, and/or at least 7 days. Such a bandage, pad, or other type of patch which can be used in the practice of the method of the present invention can take the form of an occlusive body. In practice, the occlusive body which includes the Naltrexone Hydrochloride, Naltrexol Hydrochloride, and/or Naltrexone Diol Ester is positioned on the subject's skin at the site of microneedle treatment under conditions effective to transdermally deliver the Naltrexone Hydrochloride, Naltrexol Hydrochloride, and/or Naltrexone Diol Ester to the subject's skin. Such conditions can include, for example, positioning and/or orienting the occlusive body on the skin such that the Naltrexone Hydrochloride, Naltrexol Hydrochloride, and or Naltrexone Diol Ester, when released from the occlusive body, contacts the subject's skin at a site including at least the microneedle-treated site.

### EXAMPLES

1. Materials

Naltrexone base was purchased from Mallinckrodt Inc (St. Louis, Mo.). Hanks' balanced salts modified powder, sodium bicarbonate, and propylene glycol (PG) were purchased from Sigma Chemical (St. Louis, Mo.). 4-(2-hydroxyethyl)-1-piperazineethanesulfonic acid (HEPES), gentamicin sulfate, trifluoroacetic acid (TFA), triethylamine (TEA), methanol, acetonitrile (ACN), isopropanol, hydrochloric acid (HCl), and sodium hydroxide were obtained from Fisher Scientific (Fairlawn, N.J.). 1-octane sulfonic acid, sodium salt was purchased from ChromTech (Apple Valley, Minn.).

2. Microneedle Fabrication

Microneedles (MN) were supplied by Dr. Mark Prausnitz' lab at the Georgia Institute of Technology. The arrays of 5 MN were prepared by laser etching stainless steel sheets (75 μm in thickness) and chemically polishing as described by Martanto et al., Pharm. Res. 21 (2004).

3. Synthesis procedures
3.1 Naltrexone HCl

Naltrexone base was dissolved in ACN, and 1 equimolar concentrated HCl in ACN was added dropwise. After the addition of solvent, the solution was evaporated (Büchi, Switzerland).
3.2 6-β-Naltrexol Base

The synthesis procedure was performed with minor modifications as described by Paudel et al., J. Pharm. Sci. 94 (2005) 1965-1975. After synthesis and crystallization, melting point and NMR analysis confirmed Naltrexol: 'HNMR (CDCl₃) 6.71 (d, J=8.1 Hz, 1 H), 6.56 (d, J=8.1 Hz, 1 H), 4.55 (d, J=6.1 Hz, 1 H), 3.57 (m, 1 H), (MP 185° C.).

4. Solubility of Naltrexone HCl

An excess of Naltrexone HCl (˜350 mg) was added to 1 mL of 3:1 PG:Hanks' buffer. The saturated solution was then vortexed for 1 min, sonicated for 15 min, and placed in an incubator at 32° C. for 24 h while shaking on a belly shaker. Just before filtering, a glass syringe and 0.45 μm filter were heated in an incubator to 32° C. The solution was filtered and diluted with ACN for high pressure liquid chromatography (HPLC) analysis.

5. Formulation preparation
5.1. Naltrexone Base Vs. Naltrexone HCl

A saturated solution of Naltrexone base (100 mg) was prepared in 3:1 PG:Hanks' buffer and the mixture was sonicated for 10 min and then applied onto the skin. A base equivalent of Naltrexone HCl (1.11 mg Naltrexone HCl per 1.00 mg Naltrexone base) was dissolved in the same mixture by sonication for 10 min.
5.2. Naltrexol Base pH 8.5 Vs. Naltrexol Base pH 4.5

100 mg of Naltrexol base was prepared in 3:1 H₂O:PG. The solution was briefly vortexed and sonicated for 10 min. To obtain a mixture of pH 8.5 Naltrexol base, 0.5 N sodium hydroxide was added incrementally. The same procedure was performed to obtain a pH 4.5 solution, except 0.5 M HCl was added dropwise. The ionization of Naltrexol base upon pH adjustment by HCl addition resulted in complete dissolution. The solution was saturated with excess solid when the pH was raised above the first pKa (7.4) of Naltrexol base.

6. In vitro skin diffusion studies

Hairless guinea pig skin was harvested from euthanitized animals. Animal studies were approved by the University of Kentucky IACUC. Human skin harvested during abdominoplasty was used for the diffusion studies. Human tissue use was approved by the University of Kentucky Institutional Review Board. Skin sections were obtained by removing the subcutaneous fatty tissue by scalpel dissection and stored at −20° C. A PermeGear flow-through (In-Line, Riegelsville, Pa.) diffusion cell system was used for the skin permeation studies. Skin used for microneedle treatment was placed on a wafer of polydimethylsiloxane polymer, which mimicked the underlying resistance of tissue. The skin was poked 20 times with an array containing 5 MN before mounting the skin in the diffusion cell. Untreated skin samples were simply placed in the diffusion cells. Diffusion cells were kept at 32° C. with a circulating water bath. Data was collected by using skin from a single donor with three cells for untreated formulations and three to four cells for the MN treated formulations. The receiver solution was HEPES-buffered Hanks' balanced salts with gentamicin at pH 7.4, and the flow rate was adjusted to 1.1 mL/h. Each cell was charged with 0.25 mL of the respective drug solution. Samples were collected in six-hour increments over 48 h. All samples were stored at 4° C. until processed by solid phase extraction.

The cumulative quantity of drug collected in the receiver compartment was plotted as a function of time. The flux value for a given experiment was obtained from the slope of the steady state portion of the cumulative amount of drug permeated, plotted over time. Apparent permeability coefficient values were calculated from Fick's First Law of diffusion:

$$\frac{1}{A} ( \frac{ⅆ M}{ⅆ t} ) = {J}_{s} = {K}_{p} Δ C \qquad ( 1 )$$

In Eq. 1, J_{S} is the flux at steady state (nmol/cm²/h), M is the cumulative amount of drug permeating through the skin, A is the area of the skin (0.95 cm²), Kₚ is the effective permeability coefficient in cm/h, and ΔC is the difference in concentrations of Naltrexone or Naltrexol in the donor and receiver. Sink conditions were maintained in the receiver solution for the duration of the experiment, so ΔC was approximated by the drug concentration in the donor compartment.

Theoretical dosing was computed from Eq. 2, where the Aₚ is the area of the applied patch (6.61 cm²), Kₚ is the permeability of Naltrexone in microneedle-treated human skin (7.0×10⁻⁵ cm/h) and S, the solubility (300 mg/cm³).

$$Theoretical Dose = [ {A}_{P} ( {cm}^{2} ) ] ⨯ {K}_{P} \frac{cm}{hr} ⨯ S \frac{mg}{{cm}^{3}} ⨯ 24 \frac{hr}{day} = X \frac{mg}{day} \qquad ( 2 )$$

7. Quantitative analysis

Quantitative analysis of Naltrexone by HPLC was performed with a modified assay from Hussain et al., J. Pharm. Sci. 76 (1987) 356-358.

The HPLC analysis was performed as described by Vaddi et al., Pharm. Res. 22 (2005) 758-765. The HPLC system consisted of a Waters 717plus autosampler, a Waters 1525 Binary HPLC pump, and a Waters 2487 Dual λ Absorbance detector with Waters Breeze software. A Brownlee C-18 reversed-phase Spheri-5 μm column (220×4.6 mm) with a C-18 reversed phase 7 μm guard column (15×3.2 mm) was used with the UV detector set at a wavelength of 215 nm. The mobile phase for Naltrexone was 70:30 ACN:0.1% TFA with 0.065% 1-octane sulfonic acid, sodium salt adjusted to pH 3.0 with TEA and samples were run at a flow rate of 1.5 mL/min with a run time of 5 min. The mobile phase for Naltrexol consisted of 90:10 ACN:0.1% TFA adjusted to pH 3.0 with TEA. Samples were run for 10 min at 1 mL/min as described by Paudel et al., J. Pharm. Sci. 94 (2005) 1965-1975). One hundred microliters of sample was injected onto the column for both Naltrexone and Naltrexol. Samples were analyzed within the linear range of the standard curves. Naltrexone and Naltrexol exhibited excellent linearity over the entire concentration range employed in the assays.

The drugs were extracted from the buffer samples by solid phase extraction (30mg 1 cc Oasis® HLB, Waters Corp., Milford, Mass.) as described by Vaddi et al., Pharm. Res. 22 (2005) 758-765.

Before extracting the aqueous drug samples (5 mL), the cartridge was conditioned with 1 mL of methanol and 1 mL of nanopure water. After sample loading, the cartridge was washed with 1 mL of 5% methanol and the drug was eluted with ACN and analyzed by HPLC. A similar extraction method was employed for Naltrexol, except 2 mL of methanol was used in conditioning and 1 mL of nanopure water was also used to further wash the samples after being loaded onto the cartridge. The Naltrexol was eluted with 2 mL of isopropanol and finally with 1 mL ACN. Naltrexol samples were evaporated under nitrogen in a water bath maintained at 37° C. The samples were then reconstituted with 1 mL of ACN for HPLC analysis. Highly concentrated samples of Naltrexone and Naltrexol were further diluted 10 times in ACN.

8. In vitro data analysis

The data obtained from the diffusion experiments were plotted as the cumulative amount of drug collected in the receiver compartment as a function of time. Results for Naltrexone base and HCl, and Naltrexol base pH 4.5 and 8.5 in GP skin were compiled from experiments on skin from three different animals for both compounds. The low dose Naltrexol in GP and Naltrexone HCl in human skin were compiled from one piece of skin each. The statistical analysis of data was computed with one-way ANOVA using SIGMA-STAT (SPSS, Inc., Chicago, Ill.).

9. Results and discussion

As observed in Table 1, the solubility of Naltrexone base was increased in the hydrochloride form. Similarly, addition of base to Naltrexol caused a decrease in the solubility, forming a saturated opaque solution upon addition. Whereas addition of HCl to Naltrexone enhanced the solubility by ionization of the tertiary amine at pH 4.5, forming a clear solution. Solubility values of Naltrexol can be observed in Table 1.

TABLE 1
In vitro permeation parameters for NTX base and
NTX HCl with and without microneedle treatment

| | | | | |
|---|---|---|---|---|
| | | | Perme- | |
| | Solubility in | NTX Flux | ability | |
| | 3:1 PG:Hanks | (nmol/ | (×10⁵ | Lag time |
| Drug | Buffer (mM) | cm²/h) | cm/h) | (h) |

MN GP skin

| | | | | |
|---|---|---|---|---|
| NTX Base | 43.6 | 11.80 ± 5.02 | 27.1 | 18.91 ± 4.49 |
| NTX-HCl | 794.4 | 27.16 ± 4.57 | 10.2 | 2.41 ± 3.85 |

No MN GP Skin

| | | | |
|---|---|---|---|
| NTX Base | 9.94 ± 2.75 | 22.8 | 21.22 ± 3.91 |
| NTX-HCl | 2.71 ± 1.20 | 1.02 | 22.38 ± 7.68 |

Human Skin

| | | | |
|---|---|---|---|
| NTX-HCl | 2.22 ± 0.72 | 0.837 | 23.66 ± 2.74 |
| No MN | | | |
| NTX-HCl MN | 18.54 ± 4.38 | 7.0 | 12.06 ± 4.32 |

By converting the base form of naltrexone to the water soluble hydrochloride salt, the solubility was increased by 18.2 times in 3:1 PG:Hanks' buffer. Solubilities of Naltrexol base in 3:1 H₂O:PG were determined at pH 8.5 and pH 4.5; the solution was completely solubilized at the experimental concentrations employed for MN/ionization analysis, as shown in Table 2.

TABLE 2
In vitro permeation steady state fluxes, lag times, and permeability
estimates of pH adjusted NTXOL base with and without MN treatment

| | | | | |
|---|---|---|---|---|
| | Solubility or | | | |
| | Concentration in | NTXOL Flux | Permeability | Lag time |
| Drug | 3:1 H₂O:PG (mM) | (nmol/cm²/h) | (×10⁴ cm/h) | (h) |

No MN

| | | | | |
|---|---|---|---|---|
| | High Dose | | | |
| NTXOL Base pH 4.5 | **292.40 | 6.56 ± 6.49 | 0.22 | 25.97 ± 5.50 |
| NTXOL Base pH 8.5 | 2.69 | 1.36 ± 0.91 | 5.1 | 24.34 ± 5.17 |

MN Treated

| | | | |
|---|---|---|---|
| NTXOL Base pH 4.5 | 35.07 ± 8.18 | 1.2 | 6.58 ± 5.02 |
| NTXOL Base pH 8.5 | 4.79 ± 2.62 | 17.8 | 17.53 ± 6.56 |

No MN

| | | | | |
|---|---|---|---|---|
| | Low Dose | | | |
| NTXOL Base pH 4.5 | **76.02 | 11.50 ± 4.20 | 1.5 | 27.18 ± 1.31 |
| NTXOL Base pH 8.5 | 2.69 | 8.77 ± 4.08 | 32.6 | 25.37 ± 1.10 |

MN Treated

| | | | |
|---|---|---|---|
| NTXOL Base pH 4.5 | 71.98 ± 26.38 | 9.5 | 10.33 ± 7.95 |
| NTXOL Base pH 8.5 | 15.19 ± 7.33 | 56.5 | 15.67 ± 6.96 |

**Concentration of drug in solution. Freely soluble in the ionized form.

9.1 In Vitro Permeation Studies of Naltrexone in Human and Guinea Pig Skin

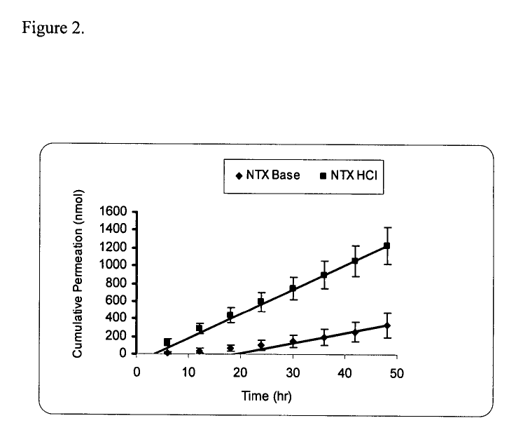

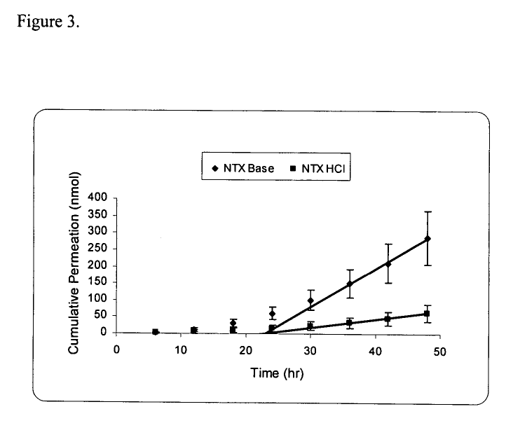

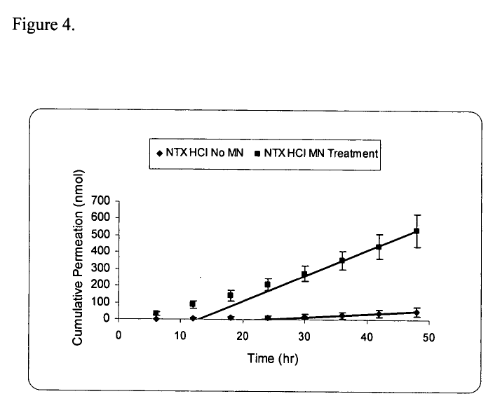

Naltrexone base and hydrochloride permeation studies with and without microneedles helped to provide insight into the effects of aqueous solubility on permeation through the aqueous pathway created by microneedle treatment. The in vitro permeation profiles from Naltrexone base and hydrochloride across GP and human skin in the presence and absence of microneedles can be observed in FIGS. 2, 3, and 4. FIG. 4 shows the comparison of MN or no MN permeation of Naltrexone HCl across human skin. Guinea pig skin from 3 animals and human skin from one donor was used to study the effects of MN treatment and aqueous solubility. There was no significant difference (p>0.05) in calculated fluxes from Naltrexone base between MN treated (11.80±5.02 nmol/cm²/h) and MN untreated (9.94±2.75 nmol/cm²/h) skin. Likewise, the lag times remained unchanged upon comparison of MN (18.91±4.49 h) and non-MN (21.22±3.91 h) treated GP skin (p>0.05), indicating that the diffusion pathway taken by the base form remains essentially the same, even when the SC has been bypassed by the MN.

Significant enhancement (p<0.05) was observed when comparing the fluxes from MN treated and untreated GP skin with Naltrexone HCl (Table 1). This finding suggests that under normal passive diffusion conditions, a more ionized water soluble compound permeates the skin at a slow rate. In contrast, when the lipid bilayer barrier is bypassed by an microneedle-created aqueous channel, significant enhancement in drug delivery is observed. This supports the fact that an alternate mechanism of permeation occurs in the presence of MN treatment. The significant change in lag time that occurs when GP skin is treated with MN provides further support of this hypothesis. When untreated, a lag time for Naltrexone HCl of 22.38±7.68 h was observed, compared to 2.41±3.85 h for MN treated GP skin (p<0.05). In order for the lag time to significantly change, a different permeation pathway must account for the observed decrease in the time to reach steady state.

Upon comparison of GP skin to human skin, flux levels were lower in human skin, as observed in a previous study (Valiveti et al., J. Control. Release 102 (2005) 509-520). However, a similar trend in the HCl MN treatment enhancement level was observed in both types of skin, 8 fold flux enhancement (p<0.05) in human skin compared to a 10-fold flux enhancement in the GP skin. Significant decreases (p<0.05) in lag times were also observed with MN treatment in both tissue types. This finding will allow us to use either human or GP skin with confidence in future in vitro studies. The human skin flux values were also used to determine if the MN treatment would allow a therapeutic dose of the drug to cross the skin at an adequate rate. Naltrexone base delivered by passive diffusion without MN should deliver a maximum daily dose of 0.4 mg for a 10 cm² patch containing Naltrexone base at its solubility limit (43.6 mM) and a permeability of 1.08×10⁻⁴ cm/h in human skin (Paudel et al., J. Pharm. Sci. 94 (2005) 1965-1975). This dose is unlikely to be therapeutic. In contrast, Naltrexone HCl delivery using MN-treated human skin and a 10 cm² patch should reach a maximum daily dose of 5.0 mg (calculated from Eq. 2) at the highest concentration measured in our studies, using the estimated permeability of 7.0×10⁻⁵ cm/h. This dose is likely to be in the therapeutic range, given that systemic absorption from an oral daily dose ranges from 2.5 to 20 mg for a 50 mg tablet (PDR, Medical Economics (1996) 2229-2233, New Jersey).
9.2. In Vitro Naltrexol Base Ionization Guinea Pig Diffusion Studies

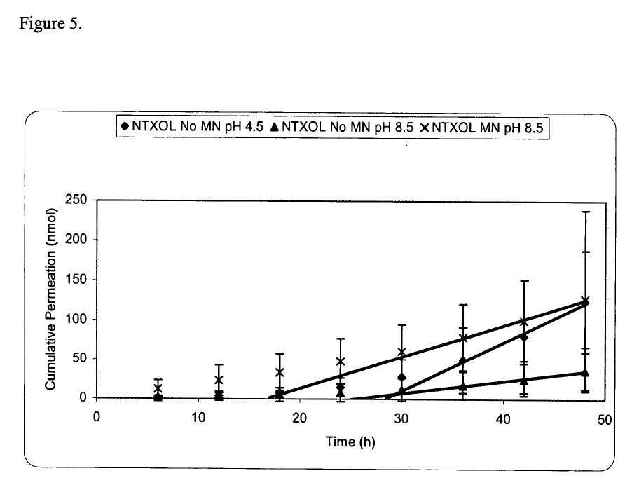

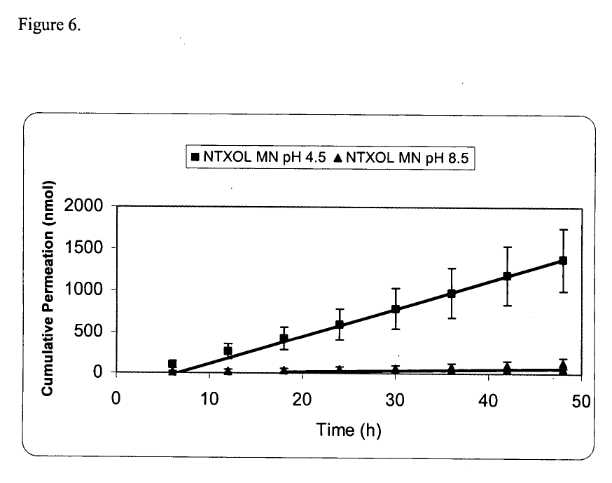

The purpose of adjusting the pH with the base form of Naltrexol was to study the effects of ionization of the tertiary amine group upon skin permeation. That is, upon ionization of Naltrexol at pH 4.5 with HCl acid, MN application should enhance permeation through GP skin. As shown in FIG. 5, there are not vast Naltrexol flux differences among the pH 8.5 MN and non-MN treated GP skin, and pH 4.5 non-MN treated skin. Thus at pH 8.5, as more of the compound is in the unionized state, solubility decreases in the predominantly aqueous formulation resulting in low flux levels. Enhancement by MN delivery is minimal, and a one-way ANOVA analysis shows that the enhancement between pH 8.5 MN treated and untreated MN skin is significant (p<0.05), but the enhancement observed is not likely to be therapeutic. Analysis of variation of the pH 4.5 compared to pH 8.5 treated and untreated skin reveals no significant enhancement between either of the two unionized forms (p>0.05). A comparison of flux values of MN treated GP skin dosed with Naltrexol pH 4.5 and Naltrexone HCl showed a slight 1.3-fold enhancement for Naltrexol, even though the two drugs are structurally very similar. It is possible that the increased hydrogen bonding potential of the third hydroxyl group of Naltrexol might be favorable for transport through MN micropores, but this needs to be investigated further.

The increased flux for ionized Naltrexol in MN-treated skin was observed at both drug concentrations tested. An earlier experiment utilizing the hydrochloride salt form of Naltrexol demonstrated lower flux rates for untreated skin (2.75±1.38 nmol/cm²/h), whereas similar results were observed for MN treated skin with Naltrexol HCl (36.12±1.77 nmol/cm²/h) versus pH 4.5 MN treated skin (35.07±8.18 nmol/cm²/h). Complete ionization of Naltrexol results in slow permeation with no MN treatment, as would be expected for a charged hydrophilic compound crossing intact stratum corneum lipid bilayers. Similarly, Naltrexol base at pH 8.5 has a very low solubility of 2.92 mM, and the flux values are also low with the MN treatment.

Calculated lag times from Naltrexol permeation profiles were consistent with the results of Naltrexone base and hydrochloride. The non-MN treated pH 4.5 and 8.5 lag times were 25.97±5.50 h and 24.34±5.17 h, respectively. On the other hand, Naltrexol pH 4.5 in the presence of MN treated skin had a lag time of only 6.58±5.02 h, which is a significant decrease in lag time (p<0.05). In order for a significant decrease in lag time to occur, the compound had to have changed its primary pathway of diffusion. Enhanced solubility by ionization provides a means to easily permeate through the MN created aqueous channels, thereby bypassing the stratum corneum.

As seen in Table 2, similar trends were observed for Naltrexol and Naltrexone. The common trend of enhanced flux and reduction in lag time remains consistent for MN treatments. The higher dose ionized Naltrexol with MN treatment provided a higher flux than the lower dose, indicating that multiple forms of drug solubilization efforts could be used to increase the flux across the skin even more. However, it was clearly evident that 26 mg/mL pH 8.5 Naltrexol low concentration diffusion cells had higher permeation rates through MN micropores at a lower degree of saturation. This observation was most likely due to less compound settling on the skin surface after charging the donor cell. At higher concentrations the degree of settling would cause the aqueous channels to become impermeable, thereby reducing the availability of an alternative pathway for diffusion. The elevated flux values at lower concentrations at low pH could be a result of a less viscous mixture or animal to animal variation in the experiment. However, the overall goal was achieved by showing that enhanced ionization resulted in higher permeation levels along with a change in the predominant diffusion pathway into the systemic circulation.
9.3 Bis(hydroxymethyl)propionic Acid Ester Prodrug of Naltrexone

The bis(hydroxymethyl)propionic acid ester prodrug of Naltrexone (“Naltrexone Diol Ester”) was synthesized as shown in FIG. 7. and described below:

Isoproylidene-2,2-bis(methoxy)propionic acid (1) was synthesized by the reaction between bis-(methoxy)propionic acid and 2,2-dimethoxypropane (Henrik et al., Macromolecules 31 (1998) 4061-4068). Compound (1) on coupling with Naltrexone (2) in the presence of DCC,DPTS gave compound (3) (Moore et al., Macromolecules 23 (1990) 65-70). Compound (3) treated with methanolic HCl gave acetonide deprotected bis(hydroxymethyl)propionic acid ester prodrug of Naltrexone (4).

A 100 mg/ml Naltrexone base equivalent of the diol ester of Naltrexone was dissolved in 3:1 propylene glycol (PG):water and was placed on MN treated GP skin for 48 hours and analyzed by HPLC. As shown in Table 3, flux levels were approximately twice that of Naltrexone HCl with MN treatment (27.16±4.57 nmol/cm2/h; data not shown), indicating a possible alternative prodrug approach for use with microneedles.

TABLE 3
In vitro permeation studies for Naltrexone
Diol Ester with MN treatment

| Drug | Flux | Lag time |
|---|---|---|
| MN Treated | (nmol/cm²/h) | (h) |
| NTX-Diol ester | 71.98 ± 26.38 | 10.33 ± 7.95 |

9.4 In Vivo Naltrexol Guinea Pig Studies.

In vivo studies of hairless guinea pigs were performed in order to obtain pharmacokinetic parameters for guinea pigs treated with 26 mg Naltrexol base gel, MN application followed by 26 mg Naltrexol base gel, 285 mg Naltrexol HCl gel and MN application followed by 285 mg Naltrexol HCl. The base was saturated in gel whereas the hydrochloride salt remained soluble in solution. Results are shown in Table 4.

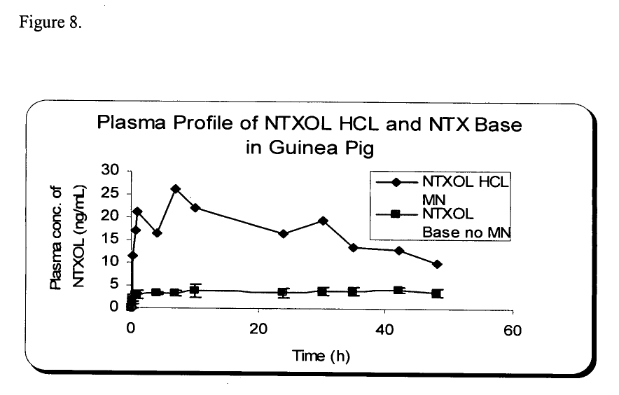

A representative plasma profile comparing Naltrexol HCL and MN treatment with Naltrexol base and no MN treatment in guinea pigs is shown in FIG. 8. As shown in the figure, plasma concentrations were markedly higher in guinea pig samples that were treated with MN and Naltrexol HCL gel therapy, as compared to those treated with no microneedles and Naltrexol base.

TABLE 4
In vivo studies of hairless guinea pigs treated with Naltrexol
base or Naltrexol HCL with or without MN treatment.

| | NTXOL | | | MN NTXOL |
|---|---|---|---|---|
| Transdermal | Base | MN NTXOL | NTXOL | HCl |
| Parameters | (n = 5) | Base | HCl | (n = 5) |
| Cₘₐₓ (ng/mL) | 1.7 ± 0.5 | 8.5 | 4.1 | 48.5 ± 18.8 |
| Tₘₐₓ (h) | 40.1 ± 11.7 | 0.7 | 7.0 | 19.2 ± 11.5 |
| AUC (ng/mL*h) | 50.5 ± 10.1 | 303.1 | 24.4 | 1349 ± 616 |
| T_{lag} (h) | 5.2 ± 1.6 | 0.7 | 1 | 0.7 ± 0.3 |
| Cₛₛ (ng/mL) | 0.95 ± 0.41 | 5.04 ± 2.57 | 1.8 ± 1.68 | 21.3 ± 6.9 |

9.5 Single Patch Doses and Gelformulations of Naltrexol HCL

For single patch dosing, a syringe containing 500 μl of Naltrexol HCl gel (21.7%) and containing a dose of about 142.5 mg can be placed onto the microneedle-treated site. The area of the patch that is to be placed over the gel to create an occlusive barrier to the external environment is suitably 6.6 cm². The reservoir of the system has a height of 2 mm from the base backing membrane. Once in place the gel will remain in contact with the skin for 72 hours upon which the affected area will be examined for irritation and cleaned. Blood samples will be subsequently taken up to 96 hours to monitor the elimination of Naltrexol HCl from each subject.

Representative gel formulations are shown in Tables 5 (gel formulation 1) and 6 (gel formulation 2).

TABLE 5
Gel Formulation 1 (weight %)

| | | |
|---|---|---|
| 21.5% | 285 | mg NTXOL-HCl |
| 75.5% | 1000 | mg of 3:1 PG:H₂O |
| 2.0% | 26.47 | mg Hydroxyethylcellulose polymer |
| 1.0% | 13.11 | mg Benzyl Alcohol |
| 100% Total | | |

For gel formulation 1,285 mg of Naltrexol HCl along with 13.11 mg of benzyl alcohol and 26.47 mg of hydroxyethylcellulose was dissolved by sonication in 1000 mg of 3:1 Propylene Glycol (PG):H₂O. The solution was sonicated for 20 minutes, briefly removing to vortex periodically. A viscous gel resulted, transparent in nature, and no visible solid particles were observed.

TABLE 6
Gel Formulation 2 (weight %)

| | | |
|---|---|---|
| 21.7% | 285 | mg NTXOL-HCl |
| 76.1% | 1000 | mg of 3:1 PG:H₂O |
| 2.0% | 25.7 | mg Hydroxyethylcellulose polymer |
| 0.15% | 1.96 | mg Propylparaben |
| 0.05% | 0.66 | mg Methylparaben |
| 100% Total | | |

For gel formulation 2,285 mg of Naltrexol HCl along with 1.96 mg Propylparaben and 0.66 mg Methylparaben was dissolved by sonication in 1000 mg of 3:1 PG:H₂O. 25.7 mg of hydroxyethylcellulose was added slowly to prevent aggregation while vortexing. The solution was sonicated for 20 minutes, briefly removing to vortex periodically. A viscous gel resulted, transparent in nature, and no visible solid particles were observed.
10. Transdermal microneedle facilitated transport of naltrexone hydrochloride 16% in healthy human volunteers.

The purpose of this study was to evaluate the pharmacokinetic parameters or efficacy of microneedle-aided delivery of a water soluble compound that, under normal circumstances, would permeate through the skin at an undesirable rate. The study is a continuation from earlier work that proved successful both in human skin in vitro (submitted to Journal of Controlled Release) and also in hairless guinea pigs in vivo. In order to perform such a study, it had to be shown that a pure, safe and effective formula could be developed to prevent any harm to the volunteers. Microneedle arrays were prepared by the lab of Dr. Mark Prausnitz at the Georgia Institute of Technology in Atlanta. In collaboration with the Center for Pharmaceutical Science and Technology a 16% naltrexone hydrochloride gel was formulated, prepared, and tested according to current good manufacturing practices as outlined by the FDA.

TABLE 7
Gel Formulation for 50 g batch

| Component | % w/w |
|---|---|
| Naltrexone HCl, USP | 16.0 |
| Sterile Water for Injection, USP | 20.25 |
| Propylene Glycol, USP | 60.75 |
| NATROSOL ® (hydroxyethyl cellulose) | 2.0 |
| Benzyl Alcohol | 1.0 |

Once the protocol received University of Kentucky IRB approval and the product was released for human testing, subjects were enrolled at the Clinical Research Center (GCRC) and each subject was dosed with a total of 400 microneedle insertions (in an array) and 2 g of gel. The dose per patch was 0.5 mg gel and 100 MN insertions for a total of 4 patches. The same procedure was followed for the control subjects minus the application of microneedle arrays. The gel patch systems were left in place on the skin for 72 hours, during which plasma samples were collected for liquid chromatography mass spectroscopy (LC-MS) analysis.

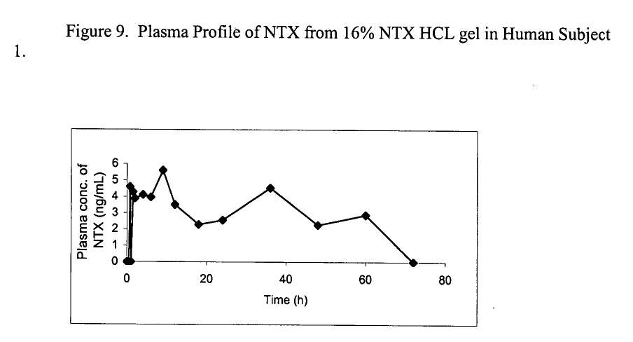

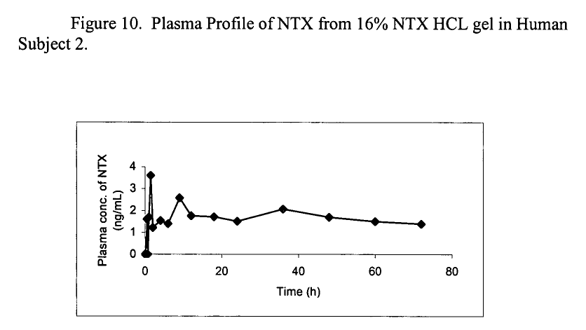

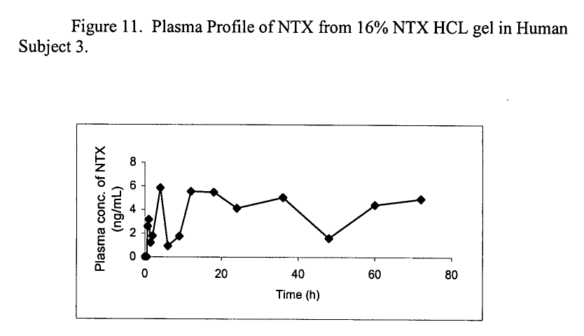

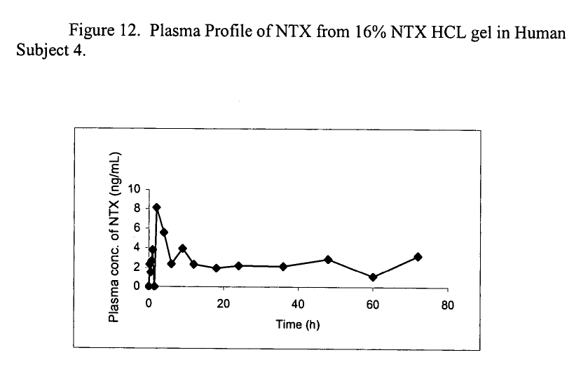

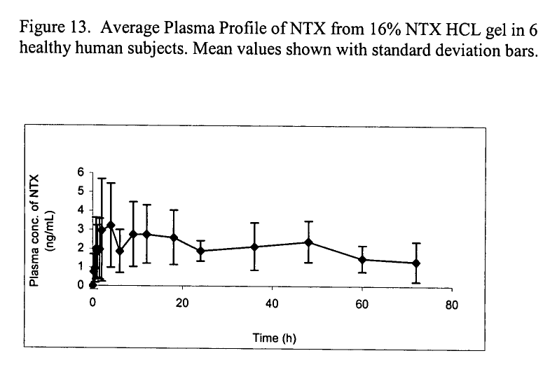

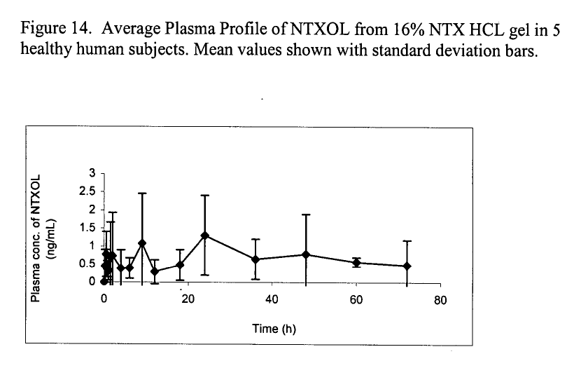

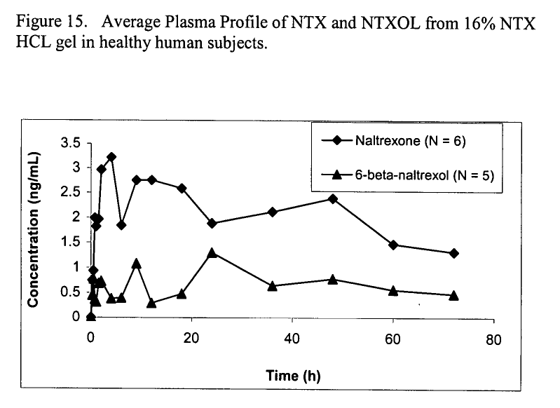

Upon evaluation of the plasma samples from the test subjects, therapeutic naltrexone (NTX) or naltrexol levels were detected by LC-MS, whereas no detectable peaks or levels of NTX or naltrexol (6-β-NTXOL, the active metabolite) were observed in the 3 control volunteers (LOQ=1 ng/mL). FIGS. 9-12 represent the plasma profiles of NTX in the microneedle (MN) treated subjects. FIGS. 13 and 14 represent the average plasma profile for NTX and for NTXOL, respectively, in MN treated subjects. FIG. 15 represents the average plasma profile for NTX and NTXOL in MN treated subjects. Tables 8 and 9 show the pharmacokinetic parameters observed in this study. Therapeutic steady state plasma NTX and NTXOL concentrations were maintained for 60-72 hours. Micropores created by MN application provided a constant rate of drug permeation.

TABLE 8
Pharmacokinetic parameters of NTX in 6 healthy
human volunteers after 72 hour application
of 16% NTX HCl gel with microneedles

| | Cₘₐₓ | Tₘₐₓ | AUC | T_{lag} | Cₛₛ |
|---|---|---|---|---|---|
| Subject | (ng/mL) | (h) | (h*ng/mL) | (h) | (ng/mL) |
| 1 | 5.6 | 9 | 194.6 | 0.5 | 3.86 |
| 2 | 3.6 | 1.5 | 122.1 | 0.25 | 1.91 |
| 3 | 5.8 | 4 | 281.1 | 0.5 | 3.4 |
| 4 | 8.1 | 2 | 178.1 | 0 | 3.61 |
| 5 | 4.4 | 48 | 132.8 | 0.5 | 1.5 |
| 6 | 2.4 | 18 | 95 | 0.25 | 1.4 |
| Mean ± | 5.0 ± 2.0 | 13.8 ± 17.9 | 147.3 ± 37.3 | 0.4 ± 0.1 | 2.5 ± 1.1 |
| S.D. | | | | | |

TABLE 9
Pharmacokinetic parameters of NTXOL in 6 healthy
human volunteers after 72 hour application
of 16% NTX HCl gel with microneedles

| | Cₘₐₓ | Tₘₐₓ | AUC | T_{lag} | Cₛₛ |
|---|---|---|---|---|---|
| Subject | (ng/mL) | (h) | (h*ng/mL) | (h) | (ng/mL) |
| 1 | 3.4 | 9 | 64.7 | 0.25 | 1.5 |
| 2 | 0.8 | 72 | 19.4 | 0.25 | 0.3 |
| 3 | 3.0 | 24 | 40.5 | 0.25 | 0.4 |
| 4 | 1.6 | 72 | 47.8 | 0.25 | 0.5 |
| 5 | 2.6 | 48 | 65.9 | 0.25 | 0.3 |
| Mean ± | 2.3 ± 1.1 | 45.0 ± 28.3 | 47.7 ± 19.2 | 0.3 ± 0.0 | 0.7 ± 0.5 |
| S.D. | | | | | |

11. In vitro/in vivo correlation of naltrexone delivered by MN enhancement in humans and human skin.

The overall goal of this study was to compare the in vitro/in vivo correlation of naltrexone (NTX) permeation rates in humans and human skin after microneedle (MN) treatment. Not only does a high correlation validate in vitro studies, it also provides a more cost efficient method to further investigate MN-enhanced permeation. MN treated human skin results from in vitro diffusion studies with a 16% NTX-HCl gel were compared to results obtained from the human subject trial. In vitro, a flux of 39.0±13.1 nmol/cm²/h was obtained, compared to the calculated flux 52.1±22.9 nmol/cm²/h which was estimated from the in vivo human steady-state plasma concentration. A 75 % correlation was observed between the human in vitro and in vivo data. No NTX was detected in control subjects where no MNs were inserted. A good correlation of human in vitro/in vivo data demonstrates that the current in vitro model is reliable and can be employed to further study MN-enhanced transdermal drug delivery.
12. Conclusions

Microneedle-assisted delivery has been shown to be a useful technique in the enhancement of flux for the transdermal candidate Naltrexone. Similarly, Naltrexone and Naltrexol are excellent molecules for use in the study of MN-assisted transdermal delivery due to their slow diffusivity in the free base form. A key mechanistic parameter of MN-assisted delivery is total drug solubility, more similar in theory to the formulation of injectable drugs, than the non-ionized lipid solubilization formulation efforts of standard passive transdermal delivery. Modifications to increase the solubility of transdermal candidates by salt formation or water soluble prodrug synthesis may be an integral part of microneedle drug selection and optimization. Reversal of the permeation pathway through aqueous MN created channels significantly enhanced the flux of the highly water soluble hydrochloride form of Naltrexone. Flux enhancement of Naltrexone HCl was greatly improved with MN treatment into the theoretical therapeutic delivery rate range in human skin. Finally, in vitro diffusion studies correlated well with data obtained from healthy individuals, furthering the validity and efficacy of MN-assisted transdermal delivery evaluated in vitro.

While the present invention has been described with reference to specific embodiments, this application is intended to cover those various changes and substitutions that may be made by those of ordinary skill in the art without departing from the spirit and scope of the appended claims.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

All publications cited herein are hereby incorporated by reference in their entirety.

We claim:

1. A method for transdermal delivery of Naltrexone Hydrochloride, Naltrexol Hydrochloride, and/or Naltrexone Diol Ester, wherein said method comprises the steps of:
(a) creating a microneedle-treated site in the skin of a subject by inserting mironeedles into the skin of said subject, followed by;
(b) applying said Naltrexone Hydrochloride, Naltrexol Hydrochloride, and/or Naltrexone Diol Ester to said microneedle-treated site.

2. The method of claim 1, wherein said microneedle-treated site is created by inserting said microneedles into and removing said microneedles from the skin of said subject prior to application of said Naltrexone Hydrochloride, Naltrexol Hydrochloride, and/or Naltrexone Diol Ester.

3. The method of claim 2, wherein said microneedles are in the form of a microneedle array.

4. The method of claim 3, wherein said microneedle array comprises 15 to 200 microneedles.

5. The method of claim 4, wherein said microneedle array comprises 50 to 100 microneedles.

6. The method of claim 2, wherein said Naltrexone Hydrochloride, Naltrexol Hydrochloride, and/or Naltrexone Diol Ester is applied to said microneedle-treated site via a topical formulation and/or by using a delivery device.

7. The method of claim 6, wherein said Naltrexone Hydrochloride, Naltrexol Hydrochloride, and/or Naltrexone Diol Ester is applied to said microneedle-treated site via a topical formulation.

8. The method of claim 7, wherein said topical formulation is a gel, a hydrogel, a topical cream, a salve, and/or an ointment.

9. The method of claim 8, wherein said topical formulation is a gel.

10. The method of claim 9, wherein said gel comprises:
(a) about 5 to about 30 weight % Naltrexone Hydrochloride, Naltrexol Hydrochloride, and/or Naltrexone Diol Ester;
(b) about 5 to about 50 weight % water;
(c) about 30 to about 70 weight % propylene glycol; and
(d) about 1 to about 5 weight % hydroxyethylcellulose polymer.

11. The method of claim 10, wherein said gel comprises about 15 to about 20 weight % Naltrexone Hydrochloride, Naltrexol Hydrochloride, and/or Naltrexone Diol Ester.

12. The method of claim 10, wherein said gel comprises about 20 weight % water.

13. The method of claim 10, wherein said gel comprises about 60 weight % propylene glycol.

14. The method of claim 10, wherein said gel comprises about 2.0 weight % hydroxyethylcellulose polymer.

15. The method of claim 10, wherein said gel comprises:
(a) about 15 to about 20 weight % Naltrexone Hydrochloride, Naltrexol Hydrochloride, and/or Naltrexone Diol Ester;
(b) about 15 to about 20 weight % water;
(c) about 50 to about 60 weight % propylene glycol; and
(d) about 2.0 weight % hydroxyethylcellulose polymer.

16. The method of claim 10, wherein said gel further comprises a preservative.

17. The method of claim 16, wherein said preservative is benzyl alcohol, propylparaben, and/or methylparaben.

18. The method of claim 9, wherein said gel further comprises a bandage, pad, and/or patch attached as a backing to the gel formulation.

19. The method of claim 8, wherein said topical formulation is a hydrogel.

20. The method of claim 19, wherein said hydrogel comprises:
(a) about 15 to about 20 weight % Naltrexone Hydrochloride, Naltrexol Hydrochloride, and/or Naltrexone Diol Ester;
(b) about 10 to about 40 weight % water; and
(c) about 35 to about 70 weight % poly(propylene glycol) acrylate, (poly(ethylene glycol) methacrylate, and/or (poly(ethylene glycol) dimethacrylate; and
(d) about 0.5 to about 5 weight % polyvinylpyrrolidone.

21. The method of claim 20, wherein said hydrogel further comprises a preservative.

22. The method of claim 21, wherein said preservative is benzyl alcohol, propylparaben, and/or methylparaben.

23. The method of claim 19, wherein said hydrogel further comprises a bandage, pad, and/or patch attached as a backing to the hydrogel formulation.

24. The method of claim 6, wherein said delivery device is a bandage, an occlusive body, and/or a patch.

25. The method of claim 24, wherein said Naltrexone Hydrochloride, Naltrexol Hydrochloride, and/or Naltrexone Diol Ester is disposed in said bandage, occlusive body, and/or patch.

26. The method of claim 25, wherein said bandage, occlusive body, and/or patch controls the release of said Naltrexone Hydrochloride, Naltrexol Hydrochloride, and/or Naltrexone Diol Ester.

27. The method of claim 24, wherein said Naltrexone Hydrochloride, Naltrexol Hydrochloride, and/or Naltrexone Diol Ester is coated on the surface of said bandage, occlusive body, and/or patch.

28. A method for treating narcotic dependence, alcohol abuse, and/or alcoholism, comprising administering to a subject in need thereof a therapeutically effective amount of Naltrexone Hydrochloride, Naltrexol Hydrochloride, and/or Naltrexone Diol Ester, wherein said Naltrexone Hydrochloride, Naltrexol Hydrochloride, and/or Naltrexone Diol Ester is administered transdermally using microneedles.

29. The method of claim 28, wherein said transdermal administration comprises the steps of:
(a) creating a microneedle-treated site in the skin of a subject by inserting mironeedles into the skin of said subject, followed by
(b) applying said Naltrexone Hydrochloride, Naltrexol Hydrochloride, and/or Naltrexone Diol Ester to said microneedle-treated site.

30. The method of claim 29, wherein said microneedle-treated site is created by inserting said microneedles into and removing said microneedles from the skin of said subject prior to application of said Naltrexone Hydrochloride, Naltrexol Hydrochloride, and/or Naltrexone Diol Ester.

31. The method of claim 30, wherein said microneedles are in the form of a microneedle array.

32. The method of claim 31, wherein said microneedle array comprises 15 to 200 microneedles.

33. The method of claim 32, wherein said microneedle array comprises 50 to 100 microneedles.

34. The method of claim 29, wherein said Naltrexone Hydrochloride, Naltrexol Hydrochloride, and/or Naltrexone Diol Ester is applied to said microneedle-treated site via a topical formulation and/or by using a delivery device.

35. The method of claim 34, wherein said Naltrexone Hydrochloride, Naltrexol Hydrochloride, and/or Naltrexone Diol Ester is applied to said microneedle-treated site via a topical formulation.

36. The method of claim 35, wherein said topical formulation is a gel, a hydrogel, a topical cream, a salve, and/or an ointment.

37. The method of claim 36, wherein said topical formulation is a gel.

38. The method of claim 37, wherein said gel comprises:
(a) about 5 to about 30 weight % Naltrexone Hydrochloride, Naltrexol Hydrochloride, and/or Naltrexone Diol Ester;
(b) about 5 to about 50 weight % water;
(c) about 30 to about 70 weight % propylene glycol; and
(d) about 1 to about 5 weight % hydroxyethylcellulose polymer.

39. The method of claim 38, wherein said gel comprises about 15 to about 20 weight % Naltrexone Hydrochloride, Naltrexol Hydrochloride, and/or Naltrexone Diol Ester.

40. The method of claim 38, wherein said gel comprises about 20 weight % water.

41. The method of claim 38, wherein said gel comprises about 60 weight % propylene glycol.

42. The method of claim 38, wherein said gel comprises about 2.0 weight % hydroxyethylcellulose polymer.

43. The method of claim 38, wherein said gel comprises:
(a) about 15 to about 20 weight % Naltrexone Hydrochloride, Naltrexol Hydrochloride, and/or Naltrexone Diol Ester;
(b) about 20 weight % water;
(c) about 60 weight % propylene glycol; and
(d) about 2.0 weight % hydroxyethylcellulose polymer.

44. The method of claim 38, wherein said gel further comprises a preservative.

45. The method of claim 44, wherein said preservative is benzyl alcohol, propylparaben, and/or methylparaben.

46. The method of claim 37, wherein said gel further comprises a bandage, pad, and/or patch attached as a backing to the gel formulation.

47. The method of claim 36, wherein said topical formulation is a hydrogel.

48. The method of claim 47, wherein said hydrogel comprises:
(a) about 15 to about 20 weight % Naltrexone Hydrochloride, Naltrexol Hydrochloride, and/or Naltrexone Diol Ester;
(b) about 10 to about 40 weight % water; and
(c) about 35 to about 70 weight % poly(propylene glycol) acrylate, (poly(ethylene glycol) methacrylate, and/or (poly(ethylene glycol) dimethacrylate; and
(d) about 0.5 to about 5 weight % polyvinylpyrrolidone.

49. The method of claim 48, wherein said hydrogel further comprises a preservative.

50. The method of claim 49, wherein said preservative is benzyl alcohol, propylparaben, and/or methylparaben.

51. The method of claim 47, wherein said hydrogel further comprises a bandage, pad, and/or patch attached as a backing to the hydrogel formulation.

52. The method of claim34, wherein said delivery device is a bandage, an occlusive body, and/or a patch.

53. The method of claim 52, wherein said Naltrexone Hydrochloride, Naltrexol Hydrochloride, and/or Naltrexone Diol Ester is disposed in said bandage, occlusive body, and/or patch.

54. The method of claim 53, wherein said bandage, occlusive body, and/or patch controls the release of said Naltrexone Hydrochloride, Naltrexol Hydrochloride, and/or Naltrexone Diol Ester.

55. The method of claim 52, wherein said Naltrexone Hydrochloride, Naltrexol Hydrochloride, and/or Naltrexone Diol Ester is coated on the surface of said bandage, occlusive body, and/or patch.

56. The method of claim 28, wherein said method comprises the step of:
(a) transdermally administering said Naltrexone Hydrochloride, Naltrexol Hydrochloride, and/or Naltrexone Diol Ester through the bore of hollow microneedles.

57. A gel formulation comprising:
(a) about 5 to about 30 weight % Naltrexone Hydrochloride, Naltrexol Hydrochloride, and/or Naltrexone Diol Ester;
(b) about 5 to about 50 weight % water;
(c) about 30 to about 70 weight % propylene glycol; and
(d) about 1 to about 5 weight % hydroxyethylcellulose polymer.

58. The formulation of claim 57, wherein said gel comprises about 15 to about 20 weight % Naltrexone Hydrochloride, Naltrexol Hydrochloride, and/or Naltrexone Diol Ester.

59. The formulation of claim 57, wherein said gel comprises about 20 weight % water.

60. The formulation of claim 57, wherein said gel comprises about 60 weight % propylene glycol.

61. The formulation of claim 57, wherein said gel comprises about 2.0 weight % hydroxyethylcellulose polymer.

62. The formulation of claim 57, wherein said gel comprises:
(a) about 15 to about 20 weight % Naltrexone Hydrochloride, Naltrexol Hydrochloride, and/or Naltrexone Diol Ester;
(b) about 15 to about 20 weight % water;
(c) about 50 to about 60 weight % propylene glycol; and
(d) about 2.0 weight % hydroxyethylcellulose polymer.

63. The formulation of claim 43, wherein said gel further comprises a preservative.

64. The formulation of claim 49, wherein said preservative is benzyl alcohol, propylparaben, and/or methylparaben.

65. A hydrogel formulation comprising:
(a) about 15 to about 20 weight % Naltrexone Hydrochloride, Naltrexol Hydrochloride, and/or Naltrexone Diol Ester;
(b) about 10 to about 40 weight % water; and
(c) about 35 to about 70 weight % poly(propylene glycol) acrylate, (poly(ethylene glycol) methacrylate, and/or (poly(ethylene glycol) dimethacrylate; and
(d) about 0.5 to about 5 weight % polyvinylpyrrolidone.

66. The hydrogel formulation of claim 65, wherein said hydrogel further comprises a preservative.

67. The hydrogel formulation of claim 66, wherein said preservative is benzyl alcohol, propylparaben, and/or methylparaben.
